(19) United States
(12) Patent Application Publication
(10) Pub. No.: US 2023/0008437 A1
(43) Pub. Date: Jan. 12, 2023

(54) Treatment Method and Treatment System

(71) Applicant: TERUMO KABUSHIKI KAISHA, Tokyo (JP)

(72) Inventors: Satoshi SAWADA, Hadano-shi (JP); Satoru SUEHARA, Kaisei-machi (JP); Keiko OTSU, Kawasaki-shi (JP)

(73) Assignee: TERUMO KABUSHIKI KAISHA, Tokyo (JP)

(21) Appl. No.: 17/933,507

(22) Filed: Sep. 20, 2022

Related U.S. Application Data
(63) Continuation of application No. PCT/JP2021/009427, filed on Mar. 10, 2021.

(30) Foreign Application Priority Data
Mar. 30, 2020 (JP) 2020-059473

(51) Int. Cl. A61N 5/06; A61B 5/00

(52) U.S. Cl. CPC A61N 5/0601; A61B 5/0071; A61N 2005/063

(57) ABSTRACT

A treatment method and a treatment system capable of treating a tumor while checking a degree of destruction of tumor cells due to emission of light and improving a treatment effect. A treatment system configured to irradiate a photosensitive substance accumulated in a tumor cell of breast cancer with excitation light, and includes: an optical device including an optical fiber configured to propagate light between a proximal portion and a distal portion of the optical device, and including, at the distal portion, an irradiation unit configured to emit light outward, and a detection unit configured to detect external light. The distal portion of the optical device is configured to be inserted into a lactiferous duct from a lactiferous duct orifice.

### CROSS-REFERENCES TO RELATED APPLICATIONS

This application is a continuation of International Application No. PCT/JP2021/009427 filed on Mar. 10, 2021, which claims priority to Japanese Application No. 2020-059473 filed on Mar. 30, 2020, the entire content of both of which is incorporated herein by reference.

### TECHNOLOGICAL FIELD

The present disclosure generally relates to a treatment method and a treatment system for destroying tumor cells.

### BACKGROUND DISCUSSION

Among treatment methods for breast cancer patients, breast conservation therapy is advantageous by being capable of improving quality of life of the patients. Meanwhile, a local recurrence after breast conservation treatment is currently observed at 10% to 20%. Therefore, local treatment in the breast conservation therapy is still not satisfactory.

In local treatment for cancer, a treatment method using a photoreactive substance is known as a method for destroying target cells such as tumor cells. In particular, in a treatment method using an antibody-photosensitive substance (hydrophilic phthalocyanine), target cells can be specifically destroyed without destroying non-target cells such as normal cells by irradiating the antibody-photosensitive substance accumulated in a tumor with excitation light (for example, near-infrared rays). Therefore, this treatment method is expected to achieve a relatively high treatment effect while minimizing side effects. Further, as the treatment effect, since an immune reaction via fragments of destroyed cells is elicited, a treatment effect by an immune function of the patient is also expected. If such local treatment using the photoreactive substance can be applied to the breast cancer patients, it is expected that a relatively high treatment effect can be achieved while conserving breasts.

U.S. Pat. No. 8,323,181 describes a device that can be inserted into a lactiferous duct to cauterize a lesion portion. The device in U.S. Pat. No. 8,323,181 destroys not only the lesion portion but also normal cells, and thus exerts a relatively heavy burden on a living body.

In order to achieve a relatively high treatment effect by a photosensitive substance, the photosensitive substance accumulated in the tumor is required to be reliably irradiated with excitation light. However, since an intensity of light is rapidly attenuated as a distance from a tissue to be transmitted increases, it is very difficult to non-invasively irradiate a tumor in a body with light having a sufficient intensity from a body surface. This requires a unit for reliably irradiating the tumor in the body with light while reducing invasiveness as much as possible. In order to maximize the treatment effect, it is required to be able to measure, during treatment, whether a reaction by the excitation light of the photosensitive substance accumulated in the tumor has progressed sufficiently. If destruction of cancer cells due to a photoreaction can be measured during the treatment, it is possible to optimally set an irradiation time and improve the treatment effect.

### SUMMARY

A treatment method and a treatment system capable of treating a tumor while checking a degree of destruction of tumor cells due to emission of light and improving a treatment effect.

A treatment method is disclosed that irradiates a photosensitive substance accumulated in a tumor cell of breast cancer with excitation light. The treatment method includes: administering the photosensitive substance into a blood vessel, a lactiferous duct, or a lymphatic vessel; inserting an optical device including an optical fiber into the lactiferous duct from a lactiferous duct orifice; irradiating the photosensitive substance accumulated in the tumor cell with the excitation light; and detecting fluorescence emitted by the photosensitive substance irradiated with the excitation light. The irradiation of the excitation light to the photosensitive substance accumulated in the tumor cell and/or the detection of the fluorescence emitted by the photosensitive substance irradiated with the excitation light is performed by the optical device inserted into the lactiferous duct.

According to the treatment method described above, the irradiation of the photosensitive substance accumulated in the tumor cell of breast cancer with the excitation light and/or the detection of the fluorescence can be performed effectively by the optical device inserted near the tumor cell. Therefore, according to this treatment method, a tumor can be treated while detecting the fluorescence to check a degree of destruction of the tumor cell due to emission of the excitation light, and a treatment effect can be improved.

The excitation light may be a near-infrared ray. The optical device may include an irradiation unit configured to emit the near-infrared ray and a detection unit configured to detect external light. The emitting of the excitation light may be performed by the irradiation unit. The detecting of the fluorescence emitted by the photosensitive substance may be performed by the detection unit. Accordingly, in the treatment method, the tumor can be treated while checking the degree of destruction of the tumor cell due to emission of the near-infrared ray, and the treatment effect can be improved.

The treatment method may further include: comparing an intensity of the fluorescence detected by the detection unit with a threshold value; and changing a position of the irradiation unit configured to emit the near-infrared ray or stopping the emission of the near-infrared ray when or after the intensity of the fluorescence reaches the threshold value. Accordingly, in the treatment method, the tumor can be treated while comparing the intensity of the fluorescence with the threshold value to check the degree of destruction of the tumor cell due to emission of the near-infrared ray with high accuracy. Therefore, the treatment method can further improve the treatment effect.

The treatment method may further include, before the emitting of the excitation light, detecting the fluorescence emitted by the photosensitive substance irradiated with the near-infrared ray while changing the position of the irradiation unit, and checking a position where the fluorescence is emitted and the intensity of the fluorescence. Accordingly, in the treatment method, the tumor cell of breast cancer can be effectively destroyed without residue as much as possible after accurately grasping a distribution of the tumor cell.

The treatment method may further include expanding a distal portion of the optical device inserted into the lactiferous duct to dispose the irradiation unit and/or the detection unit near an inner wall of the lactiferous duct. Accordingly, by reducing an influence of a body fluid in the lactiferous duct that hinders transmission of light, the irradiation of the photosensitive substance with the near-infrared ray from the irradiation unit and/or the detection of the fluorescence emitted by the photosensitive substance can be performed effectively.

In the treatment method, in the emitting of the excitation light and the detecting of the fluorescence, a breast may be deformed to be relatively thin to bring a position of the irradiation unit and/or the detection unit close to the tumor cell in which the photosensitive substance is accumulated. Accordingly, the irradiation of the photosensitive substance with the near-infrared ray from the irradiation unit and/or the detection of the fluorescence emitted by the photosensitive substance can be performed rather effectively.

The treatment method may further include, before the emitting of the excitation light, inserting a catheter for tomographic image acquisition into the lactiferous duct from the lactiferous duct orifice, and acquiring a tomographic image of a tissue including the tumor cell in which the photosensitive substance is accumulated. Accordingly, in the treatment method, the tumor cell of breast cancer can be effectively destroyed without residue as much as possible after accurately grasping the distribution of the tumor cell.

The treatment method may further include: administering a fluorescent reagent into the blood vessel, the lactiferous duct, or the lymphatic vessel, the fluorescent reagent having an excitation wavelength different from that of the photosensitive substance and being configured to emit fluorescence having a wavelength different from that of the fluorescence emitted by the photosensitive substance; and irradiating the tumor cell with light having the excitation wavelength of the fluorescent reagent and detecting the fluorescence emitted by the fluorescent reagent accumulated in the tumor cell. Even if the photosensitive substance causes a photoreaction and stops emitting the fluorescence, the fluorescent reagent can emit the fluorescence, so that an operator can rather easily recognize, by the fluorescence emitted by the fluorescent reagent that the destruction of the tumor cell progresses due to the photoreaction of the photosensitive substance.

In the treatment method, an antibody-photosensitive substance in which the photosensitive substance is bound to an antibody to be accumulated in the tumor cell may be included. Accordingly, since accumulation of the photosensitive substance in the tumor cell is improved by the antibody bound to the photosensitive substance, the tumor cell can be destroyed more reliably.

A treatment method is disclosed that irradiates a photosensitive substance with excitation light. The treatment method includes: administering the photosensitive substance into a blood vessel, a lactiferous duct, or a lymphatic vessel; inserting an optical device including an optical fiber into the lactiferous duct from a lactiferous duct orifice; irradiating the photosensitive substance in and around a target part with the excitation light; and detecting fluorescence emitted by the photosensitive substance irradiated with the excitation light.

A treatment system configured to irradiate a photosensitive substance in and around a target part with excitation light. The treatment system includes: an optical device including an optical fiber configured to propagate light between a proximal portion and a distal portion of the optical device, and including, at the distal portion, an irradiation unit configured to emit light outward; and a detection unit configured to detect external light. The distal portion of the optical device is configured to be inserted into a lactiferous duct from a lactiferous duct orifice. In accordance with an embodiment, the target part is a tumor cell of breast cancer.

According to the treatment system described above, by disposing the irradiation unit and the detection unit of the optical device near the tumor cell in the lactiferous duct, the photosensitive substance accumulated in the tumor cell can be effectively irradiated with the excitation light, and the fluorescence emitted by the photosensitive substance accumulated in the tumor cell can be detected effectively. Therefore, according to the treatment system, the tumor can be treated while detecting the fluorescence to check the degree of destruction of the tumor cell due to the emission of the excitation light, and the treatment effect can be improved.

The treatment system may further include an analysis device connected to the proximal portion of the optical device and configured to receive and analyze the light detected by the detection unit. The analysis device may be configured to calculate an intensity of fluorescence received from the detection unit, and output a threshold value reaching signal indicating that the intensity of the fluorescence is no more than a threshold value or less than the threshold value (i.e., less than or equal to the threshold value) when the intensity of the fluorescence is no more than the threshold value or less than the threshold value (i.e., less than or equal to the threshold value). Accordingly, the treatment system can notify the operator that the intensity of fluorescence is no more than the threshold value or less than the threshold value (i.e., less than or equal to the threshold value), or stop the emission of the excitation light.

The distal portion of the optical device may include an expansion portion configured to expand and contract in a radial direction. The irradiation unit and the detection unit may be disposed in the expansion portion. Accordingly, the irradiation unit and the detection unit can be disposed near the inner wall of the lactiferous duct by expanding the expansion portion in the lactiferous duct. Therefore, by reducing the influence of the body fluid in the lactiferous duct that hinders reaching of light, the photosensitive substance accumulated in the tumor cells can be effectively irradiated with the excitation light from the irradiation unit, and the fluorescence emitted by the photosensitive substance accumulated in the tumor cells can be detected effectively.

In the treatment system, an antibody-photosensitive substance in which the photosensitive substance is bound to an antibody to be accumulated in the tumor cell may be included. Accordingly, since the accumulation of the photosensitive substance in the tumor cell is improved by the antibody bound to the photosensitive substance, the tumor cell can be destroyed more reliably.

### BRIEF DESCRIPTION OF THE DRAWINGS

FIG. 1 is a plan view illustrating a treatment system according to a first embodiment.

FIG. 2 is a schematic view illustrating a state in a body when breast cancer is treated by the treatment system according to the first embodiment.

FIG. 3 is a graph illustrating intensities of light displayed on a display device.

FIG. 4 is a schematic view illustrating a state of treating the breast cancer by the treatment system according to the first embodiment.

FIG. 5 is a flowchart illustrating a flow of control in a processing unit.

FIGS. 6A and 6B are plan views illustrating a treatment system according to a second embodiment, in which FIG. 6A illustrates a state before an expansion portion is expanded, and FIG. 6B illustrates a state in which the expansion portion is expanded.

FIG. 7 is a schematic view illustrating a state in a body when breast cancer is treated by the treatment system according to the second embodiment.

FIGS. 8A and 8B are plan views illustrating a modification of the treatment system according to the second embodiment, in which FIG. 8A illustrates a state before the expansion portion is expanded, and FIG. 8B illustrates a state in which the expansion portion is expanded.

FIG. 9 is a schematic view illustrating a state in a body when breast cancer is treated by a treatment system according to a third embodiment.

FIG. 10 is a schematic view illustrating a state in a body when breast cancer is treated by a treatment system according to a fourth embodiment.

FIG. 11 is a graph illustrating intensities of light displayed on a display device.

### DETAILED DESCRIPTION

Set forth below with reference to the accompanying drawings is a detailed description of embodiments of a treatment method and a treatment system for destroying tumor cells. Note that since embodiments described below are preferred specific examples of the present disclosure, although various technically preferable limitations are given, the scope of the present disclosure is not limited to the embodiments unless otherwise specified in the following descriptions. For convenience of explanation, dimensions in the drawings may be exaggerated and may be different from actual dimensions. In the present specification and the drawings, components having substantially the same functional configuration are designated by the same reference numerals, and a duplicate description of the components having substantially the same functional configuration will be omitted. In the present specification, a side of a device to be inserted into a body lumen is referred to as a “distal side”, and a side to be operated is referred to as a “proximal side”.

### First Embodiment

A treatment system 10 according to a first embodiment is used for photoimmunotherapy in which a photosensitive substance accumulated in cell membranes of the target cells is irradiated with near-infrared rays to destroy target cells. The target cells can be tumor cells such as cancer cells. In this treatment method, an antibody-photosensitive substance, which is obtained by binding an antibody specifically accumulated only in a specific antigen on surfaces of the tumor cells and the photosensitive substance paired with the antibody, is used as a drug. The antibody is not particularly limited, and may be, for example, panitumbab, trastuzumab, HuJ591, pertuzumab, lapatinib, palbociclib, and olaparib. The photosensitive substance can be, for example, hydrophilic phthalocyanine (IR700) that reacts with near-infrared rays having a wavelength of about 700 nm, and hydrophilic phthalocyanine (IR800) that reacts with near-infrared rays having a wavelength of about 789 nm to 794 nm, but is not limited to IR700 and IR800. When IR700 receives near-infrared rays having a wavelength of about 660 nm to 740 nm, a ligand of a functional group that secures water solubility is broken, causing a structural change from water-soluble to hydrophobic. Due to this structural change, membrane protein is extracted, holes are opened in the cell membranes, and water enters the cells, so that the cancer cells can be ruptured and destroyed. IR700 is excited by receiving the near-infrared rays, and emits fluorescence having a wavelength different from an excitation wavelength. For example, IR700 emits fluorescence having a wavelength of 700 nm to 705 nm when excited by receiving near-infrared rays having a wavelength of 689 nm. A structural change of IR700 occurs while the IR700 is emitting the fluorescence by a photoreaction, and the IR700 stops emitting the fluorescence when IR700 destroyed the tumor cells and finished the role as a drug. The treatment system 10 according to the present embodiment emits the near-infrared rays to the antibody-photosensitive substance accumulated in the tumor cells, and detects a change in fluorescence emitted by the antibody-photosensitive substance, thereby measuring destruction of the tumor cells due to a photoreaction of the antibody-photosensitive substance in real time. “Real time” is not limited to a concept that the detection of a change in an intensity of the fluorescence emitted by the antibody-photosensitive substance and the irradiation with the near-infrared rays can be performed exactly at the same time, and is a broad concept that the detection is performed in parallel with the irradiation with a slight time difference, or the irradiation and the detection are repeated at short intervals, for example, of several seconds or less. The time difference may be a time lag caused by communication, calculation, or the like, or a set or calculated value. The treatment system 10 may not perform the measurement in real time as long as the destruction of the tumor cells due to the photoreaction of the antibody-photosensitive substance can be measured during treatment.

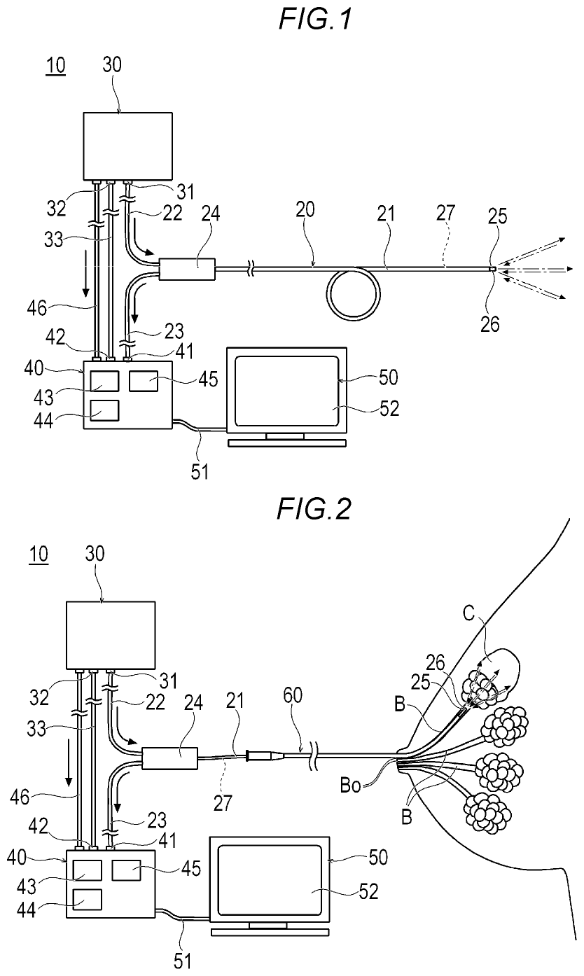

As illustrated in FIGS. 1 and 2, the treatment system 10 can include an optical device 20 that performs the irradiation and the detection of light in a lactiferous duct B, a light source device 30 that supplies light to the optical device 20, an analysis device 40 that analyzes the detected light, and a display device 50 that displays the analyzed result.

The light source device 30 can include an output unit 31 capable of outputting near-infrared rays having any wavelength with any intensity (power) or energy, and a reference light output unit 32 that outputs the same light as that of the output unit 31 as reference light. The output unit 31 is connected to the optical device 20. The reference light output unit 32 is connected to the analysis device 40. The light source device 30 outputs light to the optical device 20 such that the light can be emitted from the optical device 20 at a wavelength of, for example, 660 nm to 740 nm, and an energy of, for example, 1 Jcm⁻² to 50 Jcm⁻².

The optical device 20 can include a shaft portion 21 to be inserted into the lactiferous duct B, an input cable 22 connected to the light source device 30, an output cable 23 connected to the analysis device 40, and an optical circulator 24.

A proximal portion of the input cable 22 is connectable to the output unit 31 of the light source device 30, and a distal portion of the input cable 22 is connected to the optical circulator 24. The input cable 22 can include at least one optical fiber that propagates light, and propagates the light received from the output unit 31 to the optical circulator 24.

A proximal portion of the output cable 23 is connectable to the analysis device 40, and a distal portion of the output cable 23 is connected to the optical circulator 24. The output cable 23 includes at least one optical fiber that propagates light, and propagates the light received from the optical circulator 24 to the analysis device 40.

The shaft portion 21 includes at least one optical fiber 27 that propagates light. A proximal portion of the shaft portion 21 is connected to the optical circulator 24. A distal portion of the shaft portion 21 includes an irradiation unit 25 that emits light outward and a detection unit 26 that detects external light. Each of the shaft portion 21, the input cable 22, and the output cable 23 may include one fiber or a plurality of bundled fibers.

The optical circulator 24 propagates the light received from the input cable 22 to the shaft portion 21. The optical circulator 24 propagates the light received from the shaft portion 21 to the output cable 23. The optical device 20 may not include the optical circulator 24. For example, the shaft portion 21 may include a plurality of the optical fibers 27, the optical fiber 27 connected to the irradiation unit 25 of the shaft portion 21 may be connected to the input cable 22, and the optical fiber 27 connected to the detection unit 26 of the shaft portion 21 may be connected to the output cable 23.

The irradiation unit 25 emits outward the light propagated from a proximal side to a distal side via the optical fiber 27. The irradiation unit 25 may include, for example, a structure in which a cut end part of the optical fiber 27 is exposed, a structure in which a surface coating of the optical fiber 27 is peeled off, a lens, a diffuser, a mirror, or the like. The irradiation unit 25 can be appropriately designed such that the near-infrared rays can be emitted, for example, in a predetermined direction at a predetermined irradiation angle. The structure of the irradiation unit 25 is not limited as long as the irradiation unit 25 can emit light outward. An irradiation direction of the irradiation unit 25 (a direction in which a center of the irradiation angle is positioned) is not particularly limited. For example, the irradiation direction of the irradiation unit 25 may be a distal direction of the shaft portion 21, or a direction substantially orthogonal to an axial center of the shaft portion 21.

The detection unit 26 receives the external light into the optical fiber 27 and detects the light. The light that enters the optical fiber 27 is propagated to a proximal side of the optical fiber 27. The detection unit 26 may include, for example, a structure in which the surface coating of the optical fiber 27 is peeled off, a lens, a diffuser, a mirror, or the like. The detection unit 26 may have a structure common to that of the irradiation unit 25. That is, the irradiation unit 25 may serve as the detection unit 26.

The analysis device 40 is a device that monitors, during treatment, an action of the near-infrared rays on a tumor C including the tumor cells. The monitoring can be performed in real time, but may not be performed in real time. The analysis device 40 includes a detection light input unit 41 that receives the light detected by the detection unit 26 of the optical device 20, and a reference light input unit 42 that receives the reference light from the reference light output unit 32 of the light source device 30. The detection light input unit 41 is connected with the output cable 23 of the optical device 20. The reference light input unit 42 is connected with a reference light cable 33 connected to the reference light output unit 32 of the light source device 30.

The analysis device 40 can receive light from the output cable 23 of the optical device 20, analyze intensities of light having various wavelengths, and monitor the destruction of the tumor cells in which the antibody-photosensitive substance is accumulated.

The analysis device 40 can include, as a physical configuration of hardware, a photoelectric conversion unit 43 that converts light into an electrical signal through a filter that splits light into various wavelengths or selectively extracts only light of specific wavelengths, a storage unit 44, and a processing unit 45. The storage unit 44 can be, for example, a semiconductor memory element such as a random-access memory (RAM) or a flash memory, a hard disk, an optical disk, or the like. The storage unit 44 can write or read a threshold value T of the fluorescence, a program, and the like, which will be described later, depending on processing progress.

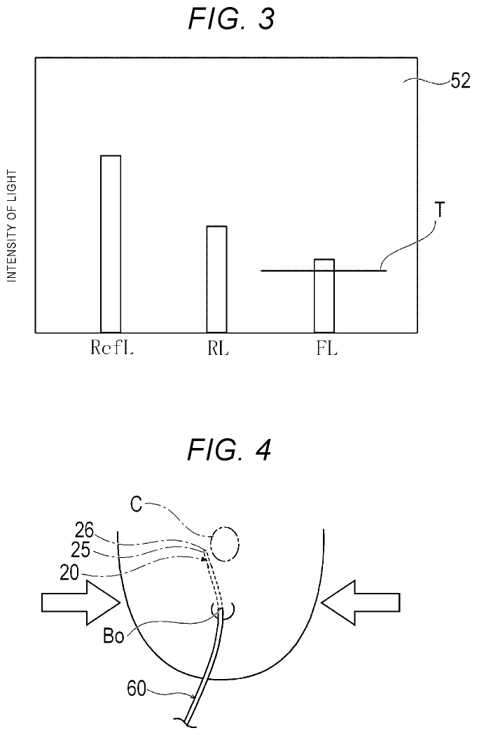

The processing unit 45 can be, for example, a central processing unit (CPU), a micro processing unit (MPU), or the like. The processing unit 45 can perform arithmetic processing by executing a program stored in the storage unit 44 by using, for example, a RAM as a work area. The processing unit 45 monitors a change in an intensity of fluorescence FL having a wavelength emitted by the antibody-photosensitive substance that receives the near-infrared rays, and when the intensity of the fluorescence FL is no more than the threshold value T or less than the threshold value T (i.e., less than or equal to threshold value T), the processing unit 45 notifies an operator via the display device 50 as illustrated in FIGS. 1 and 3. Alternatively, when the intensity of the fluorescence FL is no more than the threshold value T or less than the threshold value T (i.e., less than or equal to the threshold value T), the processing unit 45 may control the light source device 30 via a connection cable 46 connected to the analysis device 40 to stop or reduce the light output from the output unit 31 of the light source device 30. The processing unit 45 calculates an intensity of reference light RefL input to the reference light input unit 42. Further, the processing unit 45 calculates, based on the light input to the detection light input unit 41, an intensity of reflected light RL having the same wavelength as that of irradiation light (the same wavelength as that of the reference light RefL) and the intensity of the fluorescence FL having a wavelength different from those of the reference light RefL and the reflected light RL. The processing unit 45 can transmit a signal representing the calculated result to the display device 50 and display the calculated result on a display panel 52, which will be described later.

As illustrated in FIG. 1, the display device 50 is connected to the analysis device 40 via a display cable 51. The display device 50 can receive a display signal from the analysis device 40 via the display cable 51, and can display information for notifying the operator on the display panel 52. The display device 50 may include a sound output unit (speaker) for notifying the operator by sound.

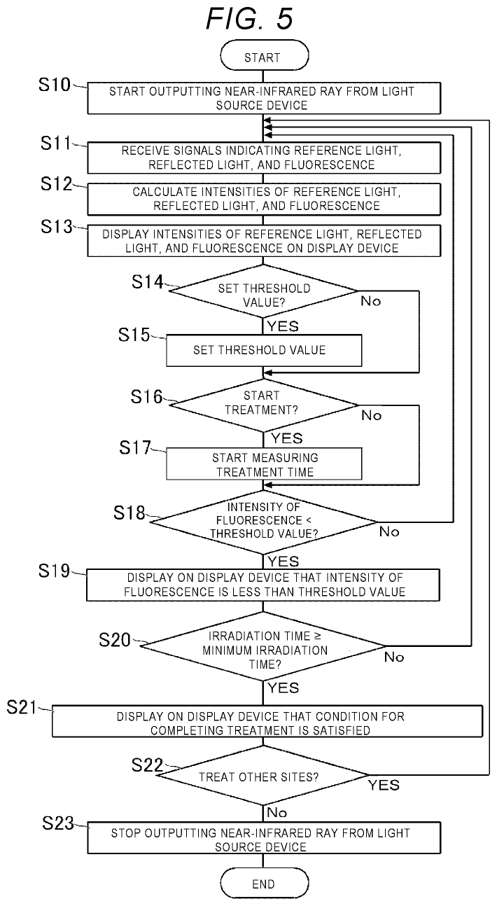

Next, an example of a treatment method of breast cancer using the treatment system 10 according to the first embodiment will be described with reference to a flowchart in the processing unit 45 illustrated in FIG. 5. The present description is not intended to limit a structure of the treatment system 10.

First, the operator administers the antibody-photosensitive substance into a blood vessel, the lactiferous duct B, or a lymphatic vessel. When administering the antibody-photosensitive substance into the blood vessel, the operator administers the antibody-photosensitive substance intravenously or intra-arterially. When administering the antibody-photosensitive substance intravenously, after approximately 12 hours to 36 hours from the administration, the operator inserts a guide wire into a lactiferous duct orifice Bo from which the guide wire can reach the lactiferous duct B disposed near the tumor C illustrated in FIG. 2. Next, the operator inserts a proximal end of the guide wire into a lumen of a catheter 60 (for example, a microcatheter), and inserts the catheter 60 into the lactiferous duct B from the lactiferous duct orifice Bo along the guide wire. Thereafter, the operator removes the guide wire from the catheter 60. When locally administering the antibody-photosensitive substance to an artery that nourishes the tumor cells, the operator waits until the antibody-photosensitive substance is accumulated in target cell membranes. When the antibody-photosensitive substance is locally administered to a nutrient artery of an organ in which the tumor C to be treated is present, the time until the antibody-photosensitive substance is accumulated in the target cell membranes is considered to be shorter than that in the case of intravenous administration, for example, about 5 minutes to 10 minutes.

Next, the operator inserts the shaft portion 21 of the optical device 20 into the catheter 60 from a proximal side of the catheter 60. A distal portion of the optical device 20 protrudes from the catheter 60 toward the distal side. Next, the operator causes the distal portion of the optical device 20 to reach a target position while checking the distal portion of the optical device 20, for example, under ultrasound fluoroscopy. The target position is a position near a target part, i.e., the tumor C such as tumor cells of breast cancer and allows irradiation of the tumor C with the near-infrared rays. When the target part is near a body surface, the position of the distal portion may be visually recognized directly from the body surface, or may be detected and checked by a high-sensitivity camera by darkening surroundings and outputting light that serves as a marker from a distal end of the optical device 20.

Next, the operator checks a treatment preparation, a treatment position, a setting of the threshold value T, and the like. Then, the operator operates the analysis device 40 that controls the light source device 30 to output the near-infrared rays from the light source device 30 (S10). The light source device 30 outputs near-infrared rays having a wavelength of, for example, 689 nm at a predetermined intensity (power) from the output unit 31 and the reference light output unit 32. The reference light RefL output from the reference light output unit 32 is input to the reference light input unit 42 of the analysis device 40. The near-infrared rays output from the output unit 31 of the light source device 30 pass through the input cable 22, the optical circulator 24, and the shaft portion 21, and are emitted toward the tumor C from the irradiation unit 25 disposed at the distal portion of the shaft portion 21. The detection unit 26 disposed at the distal portion of the shaft portion 21 detects external light. The detection unit 26 detects the reflected light RL having the same wavelength as that of the near-infrared rays (the irradiation light) emitted from the irradiation unit 25 and the fluorescence FL (700 nm to 705 nm) having a wavelength different from that of the irradiation light (or the reflected light RL) emitted by the antibody-photosensitive substance excited by receiving the near-infrared rays. The light detected by the detection unit 26 passes through the shaft portion 21, the optical circulator 24, and the output cable 23, and is input to the detection light input unit 41 of the analysis device 40. The processing unit 45 of the analysis device 40 receives signals of the reference light RefL, the reflected light RL, and the fluorescence FL (S11).

The processing unit 45 of the analysis device 40 can calculate, in real time, the intensity of the reference light RefL received by the reference light input unit 42 and the intensities of the reflected light RL and the fluorescence FL received by the detection light input unit 41 (S12). Next, as illustrated in FIG. 3, the processing unit 45 can display, in real time, the calculated intensities of the reference light RefL, the reflected light RL, and the fluorescence FL on the display panel 52 of the display device 50 (S13). The operator moves a position of the irradiation unit 25 while viewing the display panel 52, and measures the intensity and a distribution of the fluorescence FL. When setting the threshold value T or changing the threshold value T based on the measured result, the operator can operate the analysis device 40 to input the threshold value T (S14). The processing unit 45 of the analysis device 40 sets the threshold value T to the input value (S15). The threshold value T may be a predetermined absolute value to be set, a ratio of the intensity of the fluorescence FL to the intensity of the reference light RefL, or a ratio of the intensity of the detected fluorescence FL to the intensity of the reflected light RL. The threshold value T may be set in advance instead of being input by the operator during a procedure.

After measuring the intensity and the distribution of the fluorescence FL, the operator determines a treatment procedure of the tumor C (for example, division into a plurality of treatment sites and the threshold value T). Next, the operator holds the irradiation unit 25 at a position where the near-infrared rays can be emitted to a site to be treated first of the tumor C, operates the analysis device 40, and starts treatment (S16). When the operator starts the treatment, the processing unit 45 starts measuring an irradiation time (S17).

When the antibody-photosensitive substance accumulated in the tumor cells is irradiated with the near-infrared rays, the antibody-photosensitive substance causes a photoreaction to emit the fluorescence FL, and destroys the tumor cells. The antibody-photosensitive substance stops emitting the fluorescence FL after the tumor cells are destroyed. Therefore, by measuring the change in the intensity of the detected fluorescence FL in real time, a progress state of the photoreaction for destroying the tumor cells can be checked.

As described above, the processing unit 45 of the analysis device 40 can display, in real time, the calculated intensities of the reference light RefL, the reflected light RL, and the fluorescence FL on the display panel 52 of the display device 50, as illustrated in FIG. 3 (S13). The ratio of the reflected light RL to the reference light RefL is substantially constant. Therefore, one of the reference light RefL and the reflected light RL may be measured alone. The processing unit 45 determines whether the intensity of the detected fluorescence FL is less than the set threshold value T (or no more than the threshold value T) (S18). When the processing unit 45 determines that the intensity of the fluorescence FL is not less than the threshold value T (or no more than the threshold value T), the processing unit 45 determines that the progress of the photoreaction for destroying the tumor cells is insufficient, continues the output of the near-infrared rays from the light source device 30, and returns to S11. When the processing unit 45 determines that the intensity of the fluorescence FL is less than the threshold value T (or no more than the threshold value T), the processing unit 45 determines that the photoreaction for destroying the tumor cells is sufficiently performed. Next, the processing unit 45 outputs a threshold value reaching signal indicating that the intensity of the fluorescence FL is less than the threshold value T (or no more than the threshold value T), transmits the threshold value reaching signal to the display device 40, and displays the threshold value reaching signal on the display panel 52 in real time (S19).

The reason why the intensity of the fluorescence FL is less than the threshold value T (or no more than the threshold value T) is considered to include a case in which the irradiation is sufficiently performed and the photoreaction progresses, and a case in which a foreign substance such as a body fluid enters an irradiated site and the fluorescence FL cannot be detected. Therefore, the operator or the processing unit 45 may confirm that there is a certain relation among the reference light RefL, the reflected light RL, and the fluorescence FL, and start the emission of the near-infrared rays from the light source device 30. When the relation between the reference light RefL and the reflected light RL is not changed and the fluorescence FL is reduced during the emission of the near-infrared rays, the processing unit 45 determines that the emission of the near-infrared rays and the photoreaction progress stably. When the reflected light RL or the reflected light RL and the fluorescence FL is significantly reduced with respect to the reference light RefL during the emission of the near-infrared rays, the processing unit 45 determines that an irradiation state is changed due to the foreign substance. The processing unit 45 can transmit the determined result to the display device 40 and display the result on the display panel 52. As described above, a detection result of the reflected light RL may be used for determining that the stable emission of the near-infrared rays for the photoreaction progresses.

Next, the processing unit 45 determines whether the irradiation time from the start of the output of the near-infrared rays is no less than (or exceeds) a minimum irradiation time set in advance (S20). The minimum irradiation time is a lower-limit irradiation time set to ensure a lower-limit irradiation amount. Therefore, after starting the output of the near-infrared rays from the light source device 30, the processing unit 45 does not stop the irradiation until the irradiation time is no less than (or exceeds) the minimum irradiation time.

When the processing unit 45 determines that the irradiation time is less than (or does not exceed) the minimum irradiation time, the processing unit 45 continues the output of the near-infrared rays from the light source device 30 and returns to S11. When the processing unit 45 determines that the irradiation time is no less than (or exceeds) the minimum irradiation time, the processing unit 45 displays information indicating that a condition for ending the treatment of the treatment site (the emission of the near-infrared rays) is satisfied on the display device 50 in real time (S21). Accordingly, the treatment of the treatment site selected first is ended.

The minimum irradiation time may not be set. In this case, in S18, when the processing unit 45 determines that the intensity of the fluorescence FL is less than the threshold value T (or no more than the threshold value T), the processing unit 45 displays information indicating that a condition for stopping the output of the near-infrared rays is satisfied on the display device 50 in real time (S21), without performing S15 to S16. S21 may include a function of displaying that the condition for stopping the output is satisfied and temporarily stopping the output. When the output is to be temporarily stopped, the light source is stopped or at least a part of an optical path including the input cable 22 is shielded.

Next, when the treatment of the selected treatment site is ended, the operator can operate the analysis device 40 to select whether to treat other sites of the tumor C or to end the treatment of the tumor C (S22). The operator shifts and moves the irradiation unit 25 to a position where the near-infrared rays can be emitted to a next treatment site, and holds the irradiation unit 25. Thereafter, the operator starts treatment of the new treatment site (S16). Then, in the same manner as described above, the operator can measure the change in the intensity of the fluorescence FL in real time, and performs the treatment by the near-infrared rays until the condition for ending the treatment is satisfied (S21). Accordingly, the operator can sequentially treat the plurality of treatment sites. When the operator treats all the treatment sites of the tumor C and determines that there are no other treatment sites, the operator operates the analysis device 40 to select whether to end the treatment of the tumor C (S22). Accordingly, the processing unit 45 stops the output of the near-infrared rays from the light source device 30 (S23). As described above, the operator can alternately repeat the movement of the position of the irradiation unit 25 and the treatment of destroying the tumor cells by the photoreaction, thereby destroying the tumor cells distributed in a wide range. Finally, the operator removes the optical device 20 and the catheter 60 from the lactiferous duct B to end the procedure. The processing unit 45 may stop the emission of the near-infrared rays every time the treatment of a selected treatment site is ended, and may start the emission of the near-infrared rays every time the treatment of a selected treatment site is started.

If the optical device 20 has a certain degree of rigidity and can be pressed into the lactiferous duct B alone, the catheter 60 and the guide wire may not be used when inserting the optical device 20 into the lactiferous duct B. For example, the distal portion of the optical device 20 may be shaped in a manner curved to be directed in any direction in the lactiferous duct B. Alternatively, a wire-shaped protrusion may be formed at the distal portion of the optical device 20 such that the distal portion of the optical device 20 can be rather easily oriented in the lactiferous duct B.

As illustrated in FIG. 4, during the treatment, the operator may sandwich and deform a breast to bring the irradiation unit 25 and/or the detection unit 26 close to the tumor C. A direction for sandwiching the breast can be determined based on measurement results of the intensity and the distribution of the fluorescence FL of the entire tumor C measured before the treatment.

As described above, the treatment system 10 according to the first embodiment is the treatment system 10 for irradiating the antibody-photosensitive substance accumulated in the tumor cell of breast cancer with excitation light, and includes: the optical device 20 including the optical fiber 27 capable of propagating light between the proximal portion and the distal portion, and including, at the distal portion, the irradiation unit 25 capable of emitting light outward, and the detection unit 26 capable of detecting the external light. The distal portion of the optical device 20 is insertable into the lactiferous duct B from the lactiferous duct orifice Bo.

According to the treatment system 10 described above, by disposing the irradiation unit 25 and the detection unit 26 of the optical device 20 near the tumor cells in the lactiferous duct B, the antibody-photosensitive substance accumulated in the tumor cells can be effectively irradiated with the near-infrared rays, and the fluorescence FL emitted by the antibody-photosensitive substance accumulated in the tumor cells can be detected effectively. Therefore, according to the treatment system 10, the tumor can be treated while detecting the fluorescence FL to check the degree of the destruction of the tumor cells due to the emission of the near-infrared rays, and a treatment effect can be improved.

The treatment system 10 further includes the analysis device 40 connected to the proximal portion of the optical device 20 and receiving and analyzing the light detected by the detection unit 26. The analysis device 40 calculates the intensity of the fluorescence FL received from the detection unit 26, and outputs the threshold value reaching signal indicating that the intensity of the fluorescence FL is no more than the threshold value T or less than the threshold value T (i.e., less than or equal to the threshold value T) when the intensity of the fluorescence FL is no more than the threshold value T or less than the threshold value T (i.e., less than or equal to the threshold value T). Accordingly, the treatment system 10 can notify the operator that the intensity of the fluorescence FL is no more than the threshold value T or less than the threshold value T (i.e., less than or equal to the threshold value), or stop the emission of the excitation light.

The treatment method according to the present embodiment is a treatment method that irradiates the antibody-photosensitive substance accumulated in the tumor cell of breast cancer with excitation light, and includes: administering the antibody-photosensitive substance into the blood vessel, the lactiferous duct B, or the lymphatic vessel; inserting the optical device 20 including the optical fiber 27 into the lactiferous duct B from the lactiferous duct orifice Bo; irradiating the antibody-photosensitive substance accumulated in the tumor cell with the excitation light; and detecting the fluorescence FL emitted by the antibody-photosensitive substance irradiated with the excitation light. The irradiation of the excitation light to the antibody-photosensitive substance accumulated in the tumor cell and/or the detection of the fluorescence FL emitted by the antibody-photosensitive substance irradiated with the excitation light can performed by the optical device 20 inserted into the lactiferous duct B.

According to the treatment method described above, the irradiation of the antibody-photosensitive substance accumulated in the tumor cells of breast cancer with the excitation light and/or the detection of the fluorescence FL can be performed rather effectively by the optical device 20 inserted near the tumor cells. Therefore, according to this treatment method, the tumor can be treated while detecting the fluorescence FL to check the degree of destruction of the tumor cells due to the emission of the excitation light in real time, and the treatment effect can be improved.

The excitation light may be a near-infrared ray. The optical device 20 may include the irradiation unit 25 capable of emitting the near-infrared ray and the detection unit 26 capable of detecting the external light. The emitting of the excitation light may be performed by the irradiation unit 25. The detecting of the fluorescence emitted by the antibody-photosensitive substance may be performed by the detection unit 26. Accordingly, in the treatment method, the tumor can be treated while checking the degree of destruction of the tumor cells due to the emission of the near-infrared rays, and the treatment effect can be improved.

The treatment method further includes: comparing the intensity of the fluorescence FL detected by the detection unit 26 with the threshold value T; and changing the position of the irradiation unit 25 capable of emitting the near-infrared ray or stopping the emission of the near-infrared ray when or after the intensity of the fluorescence FL reaches the threshold value T. Accordingly, in the treatment method, the tumor can be treated while comparing the intensity of the fluorescence FL with the threshold value T to check the degree of destruction of the tumor cells due to the emission of the near-infrared ray with relatively high accuracy. Therefore, the treatment method can further improve the treatment effect.

The treatment method includes, before the emitting of the excitation light, detecting the fluorescence FL emitted by the antibody-photosensitive substance irradiated with the near-infrared ray while changing the position of the irradiation unit 25, and checking the position where the fluorescence FL is emitted and the intensity of the fluorescence FL. Accordingly, in the treatment method, the tumor cells of breast cancer can be effectively destroyed without residue as much as possible after accurately grasping the distribution of the tumor cells.

In the treatment method, in the emitting of the excitation light and the detecting of the fluorescence FL, the breast is deformed to be relatively thin to bring the position of the irradiation unit 25 and/or the detection unit 26 close to the tumor cell in which the antibody-photosensitive substance is accumulated. Accordingly, the irradiation of the antibody-photosensitive substance with the excitation light from the irradiation unit 25 and/or the detection of the fluorescence FL emitted by the antibody-photosensitive substance can be performed rather effectively.

### Second Embodiment

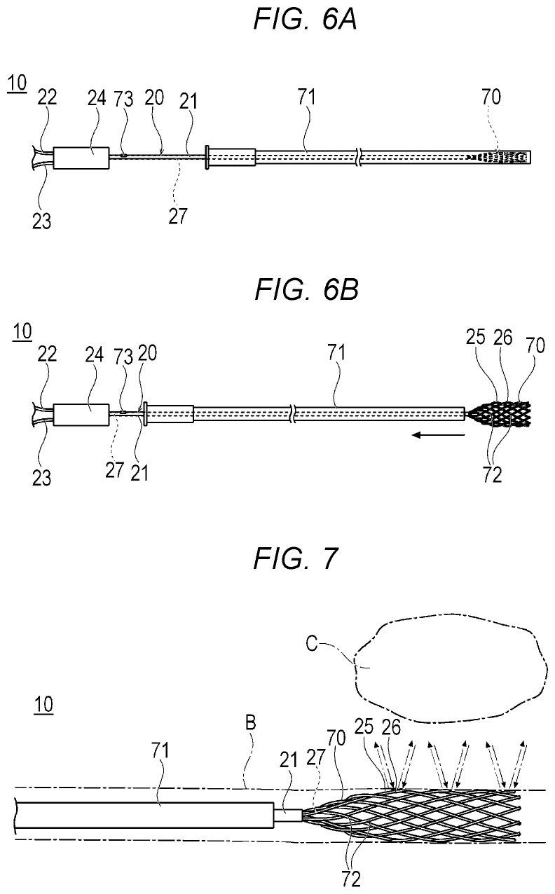

As illustrated in FIGS. 6A, 6B, and 7, the treatment system 10 according to a second embodiment is different from that of the first embodiment in that a distal portion of the shaft portion 21 of the optical device 20 is provided with an expansion portion 70 expandable and contractible in a radial direction, and a sheath 71 capable of contracting and accommodating the expansion portion 70.

The distal portion of the shaft portion 21 is connected to the expansion portion 70 expandable and contractible in the radial direction (a direction perpendicular to an axial center of the shaft portion 21). The expansion portion 70 is formed in a mesh shape by a light guide body capable of propagating light. A proximal portion of the expansion portion 70 is connected to the shaft portion 21, and a distal portion of the expansion portion 70 expands to have an outer diameter larger than an outer diameter of the shaft portion 21 in a natural state in which no external force is applied. That is, in the natural state, the expansion portion 70 has gaps due to the mesh shape, and is formed in a tubular shape such that an inner diameter and the outer diameter increase toward a distal side. In the expansion portion 70, a plurality of thin wire members 72 are knitted to form the gaps, and at the distal portion of the expansion portion 70, the plurality of wire members 72 are connected so as not to be unraveled.

The expansion portion 70 preferably has a structure in which a radial force is not applied to an inner wall of the lactiferous duct B as much as possible during expansion. Accordingly, a burden on the lactiferous duct B due to the expansion of the expansion portion 70 can be reduced. A material for implementing the expansion portion 70 can include, for example, a rubber material having relatively high stretchability, or a relatively thin and flexible thread-shaped member.

At least one of the plurality of wire members 72 forming the expansion portion 70 may be the optical fiber 27 extending from the shaft portion 21 and supplied with near-infrared rays. The optical fiber 27 forming at least a part of the expansion portion 70 includes at least one irradiation unit 25 and at least one detection unit 26 in an axial center direction of the optical fiber 27. The optical fiber 27 forming at least a part of the extension portion 70 may include a plurality of irradiation units 25 arranged in the axial center direction of the optical fiber 27, or an irradiation unit 25 formed long in the axial center direction. The optical fiber 27 forming at least a part of the extension portion 70 may include a plurality of detection units 26 arranged in the axial center direction of the optical fiber 27, or a detection unit 26 formed long in the axial center direction. A position marker 73 is disposed in the proximal portion of the optical device 20 (for example, a proximal portion of the shaft portion 21) at a position coincident with positions of the irradiation unit 25 and the detection unit 26 of the expansion portion 70 in a circumferential direction. The position marker 73 can be used for the operator to grasp the circumferential positions of the irradiation unit 25 and the detection unit 26, which are invisible (i.e., not visible) to the operator due to insertion into the lactiferous duct B.

The sheath 71 is a cylindrical member capable of accommodating the shaft portion 21 and the expansion portion 70. As illustrated in FIG. 6A, the sheath 71 can contract and accommodate the expansion portion 70 in the radial direction by moving in a distal direction with respect to the shaft portion 21 and the expansion portion 70. As illustrated in FIG. 6B, the sheath 71 releases the expansion portion 70 by moving in a proximal direction with respect to the shaft portion 21 and the expansion portion 70 from a state in which the expansion portion 70 is accommodated. Accordingly, the expansion portion 70 can be restored to an original spread shape by its own elastic force.

When using the treatment system 10 according to the second embodiment, as illustrated in FIG. 6A, the operator inserts the optical device 20 from the lactiferous duct orifice Bo into the lactiferous duct B in a state in which the expansion portion 70 is accommodated in the sheath 71. Thereafter, as illustrated in FIGS. 6B and 7, the operator moves the sheath 71 in the proximal direction to release the expansion portion 70 from the sheath 71.

As a result, the expansion portion 70 expands by its own restoring force and comes into contact with the inner wall of the lactiferous duct B, or is disposed near the inner wall of the lactiferous duct B. The irradiation unit 25 and the detection unit 26 are disposed in the expansion portion 70. Therefore, since the near-infrared rays can be emitted near the inner wall of the lactiferous duct B, it is possible to reduce an influence of a body fluid in the lactiferous duct B, which hinders reaching of the light, on emission of the light. Therefore, the near-infrared rays can be effectively emitted to an antibody-photosensitive substance accumulated in tumor cells. Since the light can be detected near the inner wall of the lactiferous duct B, it is possible to reduce an influence of the body fluid in the lactiferous duct B, which hinders the reaching of the light, on detection of the light. Therefore, the reflected light RL of the near-infrared rays and the fluorescence FL emitted by the antibody-photosensitive substance can be detected effectively by the detection unit 26. The body fluid in the lactiferous duct B can flow through the gaps of the mesh-shaped expansion portion 70. Therefore, the expansion portion 70 is likely to expand without being hindered by the body fluid and come into contact with the inner wall of the lactiferous duct B, or to be positioned near the inner wall of the lactiferous duct B.

By checking the position of the position marker 73 at the proximal portion of the optical device 20, the operator can orient the circumferential positions of the irradiation unit 25 and the detection unit 26 in a desirable direction.

As described above, in the treatment system 10 according to the second embodiment, the distal portion of the optical device 20 includes the expansion portion 70 expandable and contractible in the radial direction, and the irradiation unit 25 and the detection unit 26 are disposed in the expansion portion 70. Accordingly, the irradiation unit 25 and the detection unit 26 can be disposed near the inner wall of the lactiferous duct B by expanding the expansion portion 70 in the lactiferous duct B. Therefore, by reducing the influence of the body fluid in the lactiferous duct B that hinders the reaching of the light, the antibody-photosensitive substance accumulated in the tumor cell can be effectively irradiated with the near-infrared rays from the irradiation unit 25, and the fluorescence FL emitted by the antibody-photosensitive substance can be detected rather effectively.

A treatment method according to the second embodiment includes expanding the distal portion of the optical device 20 inserted into the lactiferous duct B to dispose the irradiation unit 25 and/or the detection unit 26 near the inner wall of the lactiferous duct B. Accordingly, by reducing the influence of the body fluid in the lactiferous duct B that hinders transmission of light, the irradiation of the antibody-photosensitive substance with the near-infrared rays from the irradiation unit 25 and/or the detection of the fluorescence FL emitted by the antibody-photosensitive substance can be performed rather effectively.

A structure of the expansion portion 70 is not particularly limited. For example, the expansion portion 70 may be a so-called self-expandable stent-like member in which a plurality of slit-shaped through holes penetrating from an outer peripheral surface to an inner peripheral surface are formed by laser processing or the like in a circular pipe that serves as a material, and the distal portion is shaped in a state of being expanded in diameter outward in the radial direction. In this case, the optical fiber 27 including the irradiation unit 25 and the detection unit 26 is fixed to the expansion portion 70 in a manner of winding around the expansion portion 70. The expansion portion 70 may be implemented by a light guide body that is not an optical fiber, and may have a structure that can receive the near-infrared rays from the optical fiber 27 forming the shaft portion 21 and can emit the near-infrared rays outward, and can receive external light and propagate the external light to the optical fiber 27.

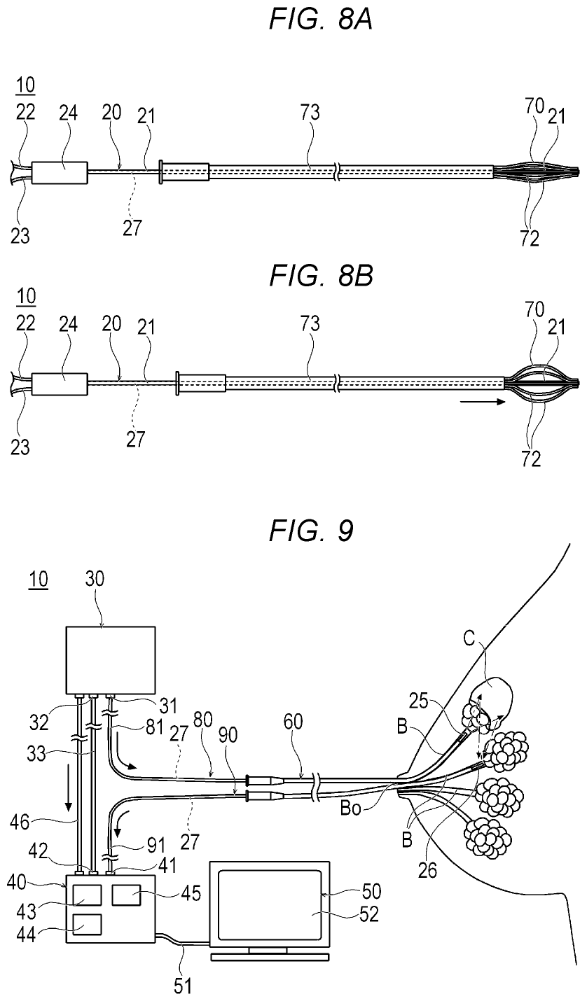

As in a modification illustrated in FIGS. 8A and 8B, the expansion portion 70 may include an outer tube 73 that accommodates the shaft portion 21 including the optical fiber 27. The distal portion of the expansion portion 70 including the plurality of wire members 72 can be fixed to the distal portion of the shaft portion 21, and the proximal portion of the expansion portion 70 can be fixed to a distal portion of the outer tube 73. The extension portion 70 can be a light guide body connected to the optical fiber 27 forming the shaft portion 21, or a part of the optical fiber 27. As illustrated in FIG. 8B, the operator can apply a compressive force in the axial center direction to the expansion portion 70 by moving the outer tube 73 in the distal direction with respect to the shaft portion 21. Accordingly, the expansion portion 70 can be expanded outward in the radial direction. As illustrated in FIG. 8A, the operator can contract the expansion portion 70 inward in the radial direction by moving the outer tube 73 in the proximal direction with respect to the shaft portion 21.

The expansion portion may be one wire member or a plurality of wire members wound in a spiral shape (a coil shape), a balloon inflated by inflowing a fluid, or the like.

### Third Embodiment

As illustrated in FIG. 9. the treatment system 10 according to a third embodiment is different from that of the first embodiment by separately including a first optical device 80 including the irradiation unit 25 and a second optical device 90 including the detection unit 26.

The first optical device 80 includes a first shaft portion 81 including the optical fiber 27 that receives near-infrared rays from the output unit 31 of the light source device 30, and the irradiation unit 25 that emits the near-infrared rays is disposed at a distal portion of the first shaft portion 81. The second optical device 90 includes a second shaft portion 91 including the optical fiber 27 that propagates light to the detection light input unit 41 of the analysis device 40, and the detection unit 26 that detects the external reflected light RL and the fluorescence FL is disposed at a distal portion of the second shaft portion 91.

When using the treatment system 10 according to the third embodiment, the operator inserts the first shaft portion 81 from the lactiferous duct orifice Bo into the lactiferous duct B to dispose the irradiation unit 25 at a position where the near-infrared rays can be emitted to an antibody-photosensitive substance accommodated in tumor cells. Thereafter, the operator inserts the second shaft portion 91 from the lactiferous duct orifice Bo into a lactiferous duct B different from the lactiferous duct B in which the irradiation unit 25 is disposed. Next, the operator disposes the detection unit 26 at a position where the fluorescence FL from the tumor cells irradiated with the near-infrared rays can be detected. Thereafter, the operator operates the analysis device 40 that controls the light source device 30 to emit the near-infrared rays from the irradiation unit 25, and detects the reflected light RL and the fluorescence FL by the detection unit 26. Accordingly, the operator can measure a change in an intensity of the fluorescence FL to be detected, for example, in real time, and can check a progress state of a photoreaction for destroying the tumor cells.

As another modification in which the first optical device 80 and the second optical device 90 are different, the first optical device 80 including the irradiation unit 25 may be inserted into the lactiferous duct B, and the second optical device 90 including the detection unit 26 may be disposed on a skin of a breast or the like outside a body. As still another example, the second optical device 90 including the detection unit 26 may be inserted into the lactiferous duct B, and the first optical device 80 including the irradiation unit 25 may be disposed on the skin of the breast or the like outside the body.

### Fourth Embodiment

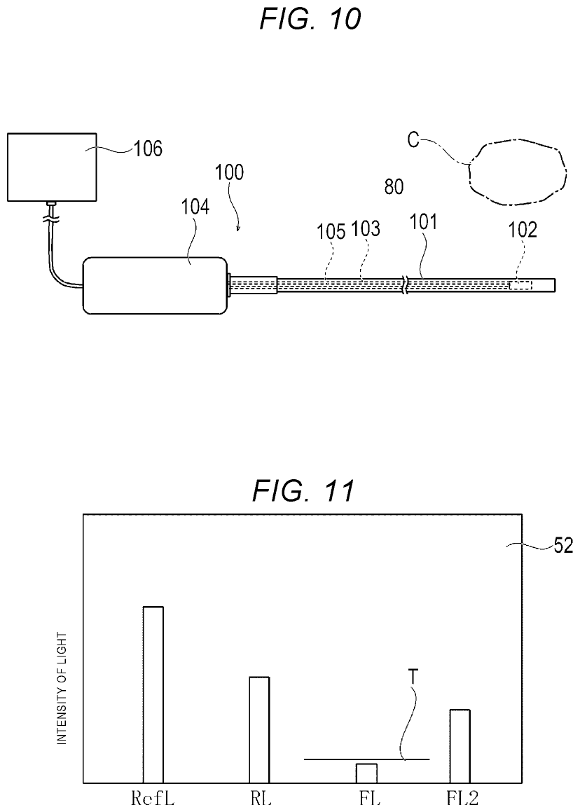

As illustrated in FIG. 10, an optical device 100 of the treatment system 10 according to a fourth embodiment may be an OCT catheter that detects reflected light and forms a tomographic image of a biological tissue by optical coherence tomography (OCT). The optical device 100 includes an elongated outer tube 101, a scanning unit 102 disposed in the outer tube 101 and serving as an irradiation unit that emits light and serving as a detection unit that detects light, a drive shaft 103 disposed in the outer tube 101 and rotationally driving the scanning unit 102, a drive source 104 that applies a rotational force to the drive shaft 103, an optical fiber 105 that is disposed inside the drive shaft 103 and rotates together with the drive shaft 103, and is connected to the scanning unit 102, and a control unit 106 connected to the optical fiber 105 to create a tomographic image. The control unit 106 includes a light source device and an analysis device. The control unit 106 controls the drive source 104 to rotate the drive shaft 103 and the scanning unit 102. The control unit 106 can cause light to emit from the scanning unit 102 and detect the reflected light to create a circumferential tomographic image surrounding the optical device 100. Accordingly, the operator can grasp a position and a distribution of the tumor C based on the tomographic image obtained from an OCT catheter 60. The operator uses the control unit 106 to cause the scanning unit 102 serving as the irradiation unit to output near-infrared rays, and to cause the scanning unit 102 serving as the detection unit to detect the reflected light RL and the fluorescence FL. Accordingly, the operator can measure destruction of tumor cells due to a photoreaction of an antibody-photosensitive substance, for example, in real time by using the OCT catheter that forms the tomographic image. At this time, since the scanning unit 102 serving as the irradiation unit and the detection unit rotates, the near-infrared rays can be output and light can be detected around the entire periphery of the optical device 100. Therefore, the optical device 100 can effectively destroy the tumor cells in a relatively wide range. The scanning unit 102 may not rotate. The scanning unit 102 may move inside the outer tube 101 in the axial center direction while rotating, thereby acquiring a three-dimensional image in a wide range in an axial center direction, and destroying the tumor cells in a wide range.

The outer tube 101 is preferably in close contact with the lactiferous duct B such that the near-infrared rays can be effectively emitted from the scanning unit 102 to the antibody-photosensitive substance accumulated in the tumor cells, and the fluorescence FL emitted by the antibody-photosensitive substance can be detected effectively by the scanning unit 102. Therefore, it can be preferable that an outer diameter of the outer tube 101 is slightly larger than an inner diameter of the lactiferous duct B, or that a probe is inserted into the lactiferous duct B in advance before the outer tube 101 is inserted.

As a catheter for tomographic image acquisition for a tissue including the tumor C, an ultrasound (IVUS) catheter may be inserted into the lactiferous duct B instead of the OCT catheter. The ultrasound catheter can acquire a tomographic image up to a position deeper than that of the OCT catheter. Since the detection by the ultrasound catheter is not performed by emitting light, the ultrasound catheter can be used in combination with the optical device 20 or the like of the treatment system 10 according to the first to third embodiments. When using ultrasound, measurement cannot be performed if air is present between an ultrasonic transducer and an observation target, and thus it is preferable to bring the ultrasound catheter into close contact with the inner wall of the lactiferous duct B. Therefore, for example, a thick probe or a balloon filled with a liquid may be disposed on a surface of the ultrasound catheter.

As described above, a treatment method according to the fourth embodiment includes, before the emitting of the near-infrared ray, inserting the catheter for tomographic image acquisition into the lactiferous duct from the lactiferous duct orifice Bo, and acquiring a tomographic image of the tissue including the tumor cell in which the antibody-photosensitive substance is accumulated. Accordingly, in the treatment method, the tumor cells of breast cancer can be effectively destroyed without residue as much as possible after accurately grasping a distribution of the tumor cells.

The disclosure is not limited to the embodiments described above, and various modifications can be made by those skilled in the art within a scope of the technical idea of the disclosure.

For example, as another example of the treatment method, a fluorescent reagent having an excitation wavelength different from that of the antibody-photosensitive substance serving as a target (for example, indocyanine green (ICG)) may be administered to a blood vessel, the lactiferous duct B, or a lymphatic vessel in advance. A timing and a position at which the fluorescent reagent is administered may be the same as or different from those of the antibody-photosensitive substance. Accordingly, not only the antibody-photosensitive substance but also the fluorescent reagent are accumulated in the tumor cells. For example, the indocyanine green is excited by light having a wavelength of 774 nm, and emits fluorescence FL2 having, for example, a wavelength of 805 nm. Therefore, the irradiation unit 25 emits light including near-infrared rays having a wavelength for exciting the antibody-photosensitive substance (for example, 689 nm) and light having a wavelength for exciting the fluorescent reagent different from that of the antibody-photosensitive substance (for example, 774 nm). Accordingly, as illustrated in FIG. 11, the processing unit 45 can calculate intensities of the reference light RefL (for example, a wavelength of 689 nm), the reflected light RL having the same wavelength as that of the near-infrared rays emitted from the irradiation unit 25 (for example, a wavelength of 689 nm), the fluorescence FL emitted by the antibody-photosensitive substance accumulated in the tumor cells (for example, a wavelength of 704 nm), and the fluorescence FL2 emitted by the fluorescent reagent accumulated in the tumor cells (for example, a wavelength of 805 nm), and display the intensities on the display device 50. The antibody-photosensitive substance stops emitting the fluorescence FL when the antibody-photosensitive substance receives the near-infrared rays and causes the photoreaction for destroying the tumor cells. Therefore, it can be difficult to specify a site where the tumor cell is present by the fluorescence FL. On the other hand, the fluorescent reagent does not cause a chemical change even if the antibody-photosensitive substance causes the photoreaction, and thus can emit the fluorescence FL2.

As another example different from the treatment system and the treatment method, photodynamic therapy (PDT) may be performed by administering in advance only a photosensitive substance represented by 5-aminolevulinic acid (ALA), Photofrin (porfimer sodium), and Laserphyrin, and emitting excitation light toward the tumor cells.

As described above, the treatment method may further include: administering the fluorescent reagent into the blood vessel, the lactiferous duct B, or the lymphatic vessel, the fluorescent reagent having an excitation wavelength different from that of the antibody-photosensitive substance and capable of emitting the fluorescence FL2 having a wavelength different from that of the fluorescence FL emitted by the antibody-photosensitive substance; and irradiating the tumor cell with light having the excitation wavelength of the fluorescent reagent and detecting the fluorescence FL2 emitted by the fluorescent reagent accumulated in the tumor cell. The fluorescent reagent emits the fluorescence FL2 even if the antibody-photosensitive substance causes the photoreaction and emission of the fluorescence FL is stopped, so that the operator can relatively easily recognize that the destruction of the tumor cells progresses due to the photoreaction of the antibody-photosensitive substance by the fluorescence FL2 emitted by the fluorescent reagent.

The detailed description above describes embodiments of a treatment method and a treatment system for destroying tumor cells. These disclosed embodiments represent examples of the treatment method and the treatment system for destroying tumor cells disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A treatment method that irradiates a photosensitive substance accumulated in a tumor cell of breast cancer with excitation light, the treatment method comprising:
administering the photosensitive substance into a blood vessel, a lactiferous duct, or a lymphatic vessel;
inserting an optical device including an optical fiber into the lactiferous duct from a lactiferous duct orifice;
irradiating the photosensitive substance accumulated in the tumor cell with the excitation light; and
detecting fluorescence emitted by the photosensitive substance irradiated with the excitation light, wherein
the irradiation of the excitation light to the photosensitive substance accumulated in the tumor cell and/or the detection of the fluorescence emitted by the photosensitive substance irradiated with the excitation light is performed by the optical device inserted into the lactiferous duct.

2. The treatment method according to claim 1, wherein
the excitation light includes a near-infrared ray;
the optical device includes an irradiation unit configured to emit the near-infrared ray and a detection unit configured to detect external light;
the emitting of the excitation light is performed by the irradiation unit; and
the detecting of the fluorescence emitted by the photosensitive substance is performed by the detection unit.

3. The treatment method according to claim 2, further comprising:
comparing an intensity of the fluorescence detected by the detection unit with a threshold value; and
changing a position of the irradiation unit configured to emit the near-infrared ray or stopping the emission of the near-infrared ray when or after the intensity of the fluorescence reaches the threshold value.

4. The treatment method according to claim 2, further comprising:
before the emitting of the excitation light, detecting the fluorescence emitted by the photosensitive substance irradiated with the near-infrared ray while changing a position of the irradiation unit, and checking a position where the fluorescence is emitted and the intensity of the fluorescence.

5. The treatment method according to claim 2, further comprising:
expanding a distal portion of the optical device inserted into the lactiferous duct to dispose the irradiation unit and/or the detection unit near an inner wall of the lactiferous duct.

6. The treatment method according to claim 2, wherein
in the emitting of the excitation light and the detecting of the fluorescence, a breast is deformed to be thin to bring a position of the irradiation unit and/or the detection unit close to the tumor cell in which the photosensitive substance is accumulated.

7. The treatment method according to claim 1, further comprising:
before the emitting of the excitation light, inserting a catheter for tomographic image acquisition into the lactiferous duct from the lactiferous duct orifice, and acquiring a tomographic image of a tissue including the tumor cell in which the photosensitive substance is accumulated.

8. The treatment method according to claim 1, further comprising:
administering a fluorescent reagent into the blood vessel, the lactiferous duct, or the lymphatic vessel, the fluorescent reagent having an excitation wavelength different from that of the photosensitive substance and being configured to emit fluorescence having a wavelength different from that of the fluorescence emitted by the photosensitive substance; and
irradiating the tumor cell with light having the excitation wavelength of the fluorescent reagent and detecting the fluorescence emitted by the fluorescent reagent accumulated in the tumor cell.

9. The treatment method according to claim 1, wherein
the photosensitive substance is an antibody-photosensitive substance in which the photosensitive substance is bound to an antibody to be accumulated in the tumor cell.

10. The treatment method according to claim 1, wherein the photosensitive substance is hydrophilic phthalocyanine.

11. A treatment method that irradiates a photosensitive substance with excitation light, the treatment method comprising:
administering the photosensitive substance into a blood vessel, a lactiferous duct, or a lymphatic vessel;
inserting an optical device including an optical fiber into the lactiferous duct from a lactiferous duct orifice;
irradiating the photosensitive substance in and around a target part with the excitation light; and
detecting fluorescence emitted by the photosensitive substance irradiated with the excitation light.

12. The treatment method according to claim 11, wherein the irradiation of the photosensitive substance with the excitation light is performed with the optical device inserted into the lactiferous duct.

13. The treatment method according to claim 11, wherein the detection of the fluorescence emitted by the photosensitive substance irradiated with the excitation light is performed by the optical device inserted into the lactiferous duct.

14. The treatment method according to claim 11, wherein
the optical device includes an irradiation unit configured to emit a near-infrared ray and a detection unit configured to detect external light;
the emitting of the excitation light is performed by the irradiation unit; and
the detecting of the fluorescence emitted by the photosensitive substance is performed by the detection unit.

15. The treatment method according to claim 14, further comprising:
comparing an intensity of the fluorescence detected by the detection unit with a threshold value; and
changing a position of the irradiation unit configured to emit the near-infrared ray or stopping the emission of the near-infrared ray when or after the intensity of the fluorescence reaches the threshold value.

16. A treatment system configured to irradiate a photosensitive substance in and around a target part with excitation light, the treatment system comprising:
an optical device including an optical fiber configured to propagate light between a proximal portion and a distal portion of the optical device, and including, at the distal portion, an irradiation unit configured to emit light outward; and
a detection unit configured to detect external light, wherein the distal portion of the optical device is configured to be inserted into a lactiferous duct from a lactiferous duct orifice.

17. The treatment system according to claim 16, wherein the target part is a tumor cell of breast cancer.

18. The treatment system according to claim 16, further comprising:
an analysis device connected to the proximal portion of the optical device and configured to receive and analyze the light detected by the detection unit; and
wherein the analysis device is configured to calculate an intensity of fluorescence received from the detection unit, and output a threshold value reaching signal indicating that the intensity of the fluorescence is no more than a threshold value or less than the threshold value when the intensity of the fluorescence is no more than the threshold value or less than the threshold value.

19. The treatment system according to claim 16, wherein
the distal portion of the optical device includes an expansion portion configured to expand and contract in a radial direction; and
the irradiation unit and the detection unit are disposed in the expansion portion.

20. The treatment system according to claim 16, wherein the photosensitive substance is an antibody-photosensitive substance in which the photosensitive substance is bound to an antibody to be accumulated in the tumor cell.
